(19) United States
(12) Patent Application Publication
(10) Pub. No.: US 2024/0245482 A1
(43) Pub. Date: Jul. 25, 2024

(54) INTRAORAL APPARATUS AND METHOD OF USE

(71) Applicant: Weiye Lin, Everett, WA (US)

(72) Inventor: Weiye Lin, Everett, WA (US)

(21) Appl. No.: 18/159,416

(22) Filed: Jan. 25, 2023

(51) Int. Cl. A61B 90/00; A61F 5/01

(52) U.S. Cl. CPC A61B 90/02; A61F 5/01

(57) ABSTRACT

An apparatus for preventing nasolabial folds (smile lines) comprises an upstanding wall having a first surface facing a user's lip, the upstanding wall having two lobes positioned apart on either side of the upstanding wall, wherein the lobes extend upward from a top portion of the upstanding wall to an area where an upper lip and a gum join, wherein the lobes are of a size and thickness to lift a skin between each corner of a nose and each corner of the lip above from the gum, and wherein the first surface bulges outward at locations of the lobes; and a tray having a second surface facing occlusal surfaces of lower teeth; wherein the upstanding wall and the tray define a third surface having recesses for receiving upper teeth.

### FIELD

This disclosure describes devices and methods that can prevent or reduce nasolabial folds.

### BACKGROUND

Nasolabial folds are the creases that extend from either side of your nose to the corners of your mouth. Nasolabial folds are often referred to as smile lines or laugh lines since they are most prominent when you are smiling or laughing. The decrease of cheek fat volume or the sagging of loose skin can contribute to the development of nasolabial folds. Existing therapies include the injection of fillers, fat transfer, and subcision surgeries, all of which involve invasive or surgical procedures. These invasive procedures often result in bruising, bleeding, and swelling at the treatment sites. Surgical procedures could leave scars and take longer to heal. These treatments are not permanent and often result in an unnatural appearance. Additionally, they are expensive, making long-term treatments unaffordable. Therefore, there is a need for non-invasive and affordable options for preventing or reducing nasolabial folds.

### BRIEF SUMMARY OF THE INVENTION

Devices and methods as described herein relate to preventing or reducing nasolabial folds. These devices, methods, and techniques allow a user to apply an apparatus within a user's month on a regular basis for the right amount of time.

According to embodiments, an apparatus for reducing nasolabial folds is described. The apparatus comprises an upstanding wall having a first surface facing a user's lip, the upstanding wall having two lobes positioned apart on either side of the upstanding wall, wherein the lobes extend upward from a top portion of the upstanding wall to an area where an upper lip and a gum join, wherein the lobes are of a size and thickness to lift a skin between each corner of a nose and each corner of the lip above from the gum, and wherein the first surface bulges outward at locations of the lobes; and a tray having a second surface facing occlusal surfaces of lower teeth; wherein the upstanding wall and the tray define a third surface having recesses for receiving upper teeth.

According to embodiments, the apparatus further comprises a wire positioned within the upstanding wall, wherein the wire is of a substantially same length as the upstanding wall.

According to embodiments, each of the lobes spans between first premolar and first molar of the upper teeth, or between canine and first molar of the upper teeth.

According to embodiments, the lobes have a thickness between 0.1 cm to 3 cm.

According to embodiments, the lobes have a thickness between 1 cm to 2 cm.

According to embodiments, each of the lobes has a height between 1.5 cm to 3 cm from a top portion to a bottom portion of the upstanding wall.

According to embodiments, an apparatus for reducing nasolabial folds is described. The apparatus comprises an upstanding wall having a first surface facing a user's lip, the upstanding wall having two lobes positioned apart on either side of the upstanding wall, wherein the lobes extend upward from a top portion of the upstanding wall to an area where an upper lip and a gum join, wherein the lobes are of a size and thickness to lift a skin between each corner of nose and each corner of the lip above from the gum, and wherein the first surface bulges outward at locations of the lobes; and a handle coupled to a front portion of the upstanding wall.

According to embodiments, a method of reducing nasolabial folds is described. The method comprises administrating an intraoral apparatus configured to be worn daily. The intraoral apparatus comprises an upstanding wall having a first surface facing a user's lip and a second surface facing upper teeth, the upstanding wall having two lobes positioned apart on either side of the upstanding wall, wherein the lobes extend upward from a top portion of the upstanding wall to an area where an upper lip and a gum join, wherein the lobes are of a size and thickness to lift a skin between each corner of nose and each corner of the lip above from the gum, and wherein the first surface bulges outward at locations of the lobes.

The foregoing summary is illustrative only and is not intended to be in any way limiting. Features from any of the disclosed embodiments can be used in combination with one another, without limitation. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows a side perspective view depicting an example intraoral apparatus, according to some embodiments.

FIG. 2 shows a back perspective view depicting the example intraoral apparatus of FIG. 1, according to some embodiments.

FIG. 3 shows a side perspective view depicting another example intraoral apparatus, according to some embodiments.

FIG. 4 shows a back perspective view depicting the example intraoral apparatus of FIG. 3, according to some embodiments.

FIG. 5 shows a top view depicting the example intraoral apparatus of FIG. 1, according to some embodiments.

FIG. 6 shows a cross sectional view along line 6-6 of the example intraoral apparatus of FIG. 5, according to some embodiments.

FIG. 7 shows a side view depicting the example intraoral apparatus of FIG. 1, according to some embodiments.

### DETAILED DESCRIPTION

In overview, this disclosure relates to apparatuses and methods for preventing the formation of nasolabial folds, slowing down the deepening and reducing the appearance of nasolabial folds. Nasolabial folds are the creases that extend from either side of your nose to the corners of your mouth. Nasolabial folds are often referred to as smile lines or laugh lines since they are most prominent when you are smiling or laughing. The decrease of cheek fat volume or the sagging of loose skin can both contribute to the development of nasolabial folds. The disclosure provides non-invasive, inexpensive, disposable, and low-risk approaches for preventing or reducing nasolabial folds.

As we age, we lose bone structure on our faces. Active lines and fine lines can get deeper even when we are sleeping. Once the bone structure is gone, the distance between layers of muscle increases, and the increased elevation difference between muscles alone can cause the deepening and formation of the nasolabial folds. The disclosed apparatuses are designed to allow an ever foundation to enable the muscle of facial expression, especially the orbicularis oris, zygomaticus and levator labii superioris alaeque nasi, to an even plane to reduce the formation of the nasolabial lines at the resting state of muscles. The formation of such lines is mainly due to the musculodermal insertion of lip elevator muscles. When these muscles contract, a nasolabial line is formed. This device also acts as a muscle memory modulator to reduce the activation of facial expression during sleeping, hence reducing the formation and deepening of the nasolabial folds. As a result, muscles of facial expression involved will have better resting and healing during sleeping and hence reduce the appearance of nasolabial fold over time.

In the following detailed description, reference is made to the accompanying drawings, which form a part hereof. In the drawings, similar symbols typically identify similar components unless context dictates otherwise. The illustrative embodiments described in the detailed description, drawings, and claims are not meant to be limiting. Other embodiments may be utilized, and other changes may be made, without departing from the spirit or scope of the subject matter presented here. In addition to other locations, defined terms may be found at the end of this Detailed Description.

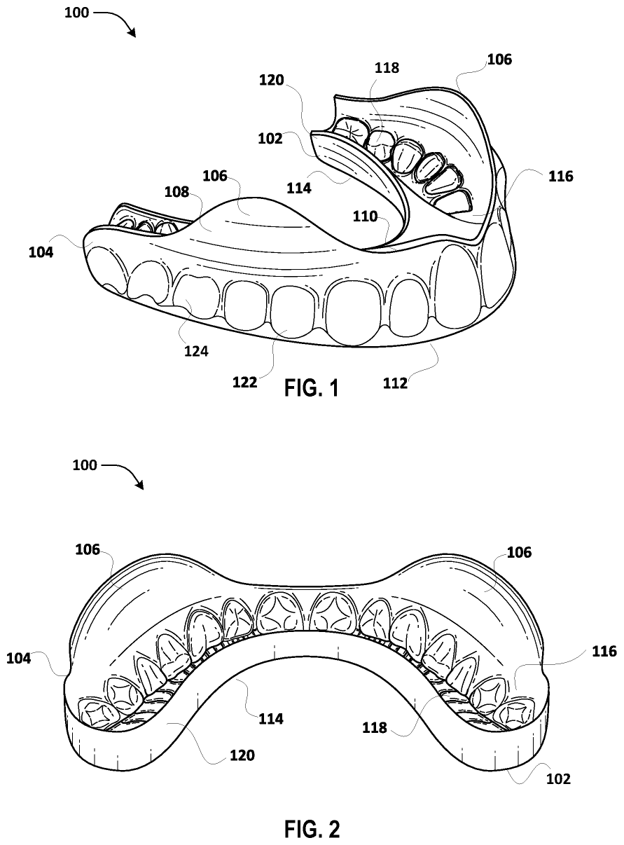

FIG. 1 shows a side perspective view depicting an example intraoral apparatus 100, according to some embodiments. The intraoral apparatus 100 has a tray 102 covering the occlusal surfaces of a user's upper teeth and an upstanding wall 104 bracing the user's upper teeth. The upstanding wall 104 further has a top portion 110 and a bottom portion 112 that define a height of the upstanding wall 104. The upstanding wall 104 has two lobes 106 positioned apart on either side of the upstanding wall 104. The lobes 106 extend upward from the top portion 110 of the upstanding wall 104 and can extend up to an area where an upper lip and a gum join, i.e., a maxillary labial frenulum or lip frenulum.

The upstanding wall 104 has a generally uniform thickness of an area covering the buccal surfaces of the upper teeth (i.e., the area between the top portion 110 and the bottom portion 112 of the upstanding wall 104), whereas the lobes 106 have a greater thickness than the upstanding wall area covering the buccal surfaces of the upper teeth. The upstanding wall 104 has a first surface 108 bulged out at the locations of the lobes 106.

The lobes 106 are configured to lift a skin between each corner of the user's nose and the user's lip to reduce or minimize the visual appearance of the nasolabial folds. The lobes 106 may have different heights and widths suitable for different sizes of mouths. The height of each of the lobes 106 is understood as the distance from the top portion 110 of the upstanding wall 104 to a top of each of the lobes 106. For instance, the lobes 106 may have a height from 0.1 cm to 3 cm. In a preferred embodiment, the lobes 106 have a height between 1 cm to 2 cm. The width of each of the lobes 106 is understood as the distance between its up curve and its down curve along the span of the upstanding wall. The width of each of the lobes 106 may be among 3 cm to 5 cm. In a preferred embodiment, each of the lobes 106 covers a gum area starting from first premolar 122 to first molar 124. The curve of the lobes 106 may start rising between canine and the first premolar and going down between the first molar and the second molar. The lobes 106 on each side usually have the same height and same width, unless the user's nasolabial folds on both sides are significantly uneven.

The tray 102 has a second surface 114 facing occlusal surfaces of lower teeth. The upstanding wall 104 and tray 102 define a third surface 116 having recesses 118 for receiving the upper teeth. The recesses 118 are custom fit for each user's upper teeth. The tray 102 may have an inner wall 120 to secure the application of the intraoral apparatus 100 to the upper jaw.

FIG. 2 shows a back perspective view depicting the example intraoral apparatus 100, according to some embodiments. The tray 102 and the upstanding wall 104 should have an appropriate length that covers the user's upper teeth. The intraoral apparatus 100 may be made of an integrated material suitable to be disposed of inside the user's mouth. The material should have an appropriate softness not to cause discomfort and sufficient rigidness to hold up the shape of the apparatus. Polymeric materials, such as PET, PETG, HDPE, PP, PVC, acrylic, and thermoplastic polymer, can be used. The intraoral apparatus 100 may be customed made in a lab setting to fit the user's dentitions. In some embodiments, the lobes 106 may be made from a material different from the tray 102 and the upstanding wall 104.

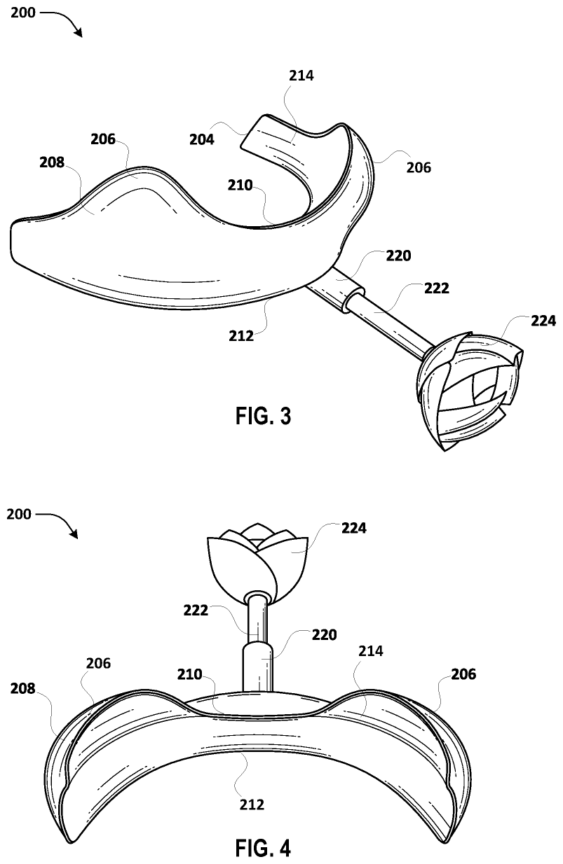

FIG. 3 shows a side perspective view depicting an intraoral apparatus 200, according to some embodiments. The intraoral apparatus 200 has an upstanding wall 204 bracing the user's upper teeth. The upstanding wall 204 further has a top portion 210 and a bottom portion 212 that define a height of the upstanding wall 204. The upstanding wall 204 has a first surface 208 facing the user's cheek and a second surface 214 facing the buccal surfaces of the upper teeth. The upstanding wall 204 has two lobes 206 positioned on either side of the upstanding wall 204. The lobes 206 extend upward from the top portion 210 of the upstanding wall 204 and can extend up to an area where an upper lip and a gum join, i.e., a maxillary labial frenulum or lip frenulum.

The upstanding wall 204 has a generally uniform thickness of an area covering the buccal surfaces of the upper teeth (i.e., the area between the top portion 210 and the bottom portion 212 of the upstanding wall 204), whereas the lobes 206 have a greater thickness than the upstanding wall area covering the buccal surfaces of the upper teeth. The first surface 208 is bulged out at the locations of the lobes 206. The lobes 206 are configured to lift a skin between the corner of a nose and the lip to reduce or minimize the appearance of the nasolabial folds. The lobes 206 may have different heights and widths suitable for different sizes of mouths. The height of each of the lobes 206 is understood as the distance from the top portion 210 of the upstanding wall 204 to a top of each of the lobes 206. For instance, the lobes 206 may have a height from 0.1 cm to 3 cm. In a preferred embodiment, the lobes 206 have a height between 1 cm to 2 cm. The width of each of the lobes 206 is understood as the distance between the up curve and the down curve of the lobes 206 along the length of the upstanding wall. The width of each of the lobes 206 may be among 3 cm to 5 cm. In a preferred embodiment, each of the lobes 206 covers a gum area starting from first premolar to first molar. The curve of the lobes 206 may start rising between canine and the first premolar and going down between the first molar and the second molar. The lobes 206 on each side usually have the same height and same width, unless the user's nasolabial folds on both sides are significantly uneven.

The intraoral apparatus 200 has a handle 220 connected to the bottom portion 212 of the upstanding wall 204. In a preferred embodiment, the handle 220 is located near a mid-line of the intraoral apparatus 200 to help with alignment. The handle 220 may take the form of a flat tab or a substantially cylindrical shape extending from the upstanding wall 204. A rod 222 with an attachment 224 may be connected to the handle 220. The rod 222 may be extendable. The attachment 224 is intended to help the user insert and remove the intraoral apparatus 200 from the mouth by providing a grip. Meanwhile, the attachment 224 may be ornamental and have different shapes. In the embodiment as shown in FIG. 3, the attachment 224 has a flower-like shape. In some embodiments, the attachment 224 may be a nob, a flange, or a flap.

FIG. 4 shows a back perspective view depicting the example intraoral apparatus 200, according to some embodiments. The upstanding wall 204 should have an appropriate length that covers the user's upper teeth. In an example embodiment, the intraoral apparatus 200 may come in three sizes that differ in the thickness of the lobes 206, the height of the lobes 206 on either side and the length of the upstanding wall 204. To decide which size fits best, the user can measure the distance between two canines of the upper teeth.

Unlike the intraoral apparatus 100, the second surface 214 of the intraoral apparatus 200 does not have recesses for receiving the upper teeth. The second surface 214 is a substantially smooth surface that fits the user's dental arch.

The intraoral apparatus 200 may be made of an integrated material suitable to be disposed of inside the user's mouth. The material should have an appropriate softness not to cause discomfort and sufficient rigidness to hold up the shape of the apparatus. Possible materials include polysiloxane and polyurethane. The intraoral apparatus 200 may be customed made in a lab setting to fit the user's dentitions. In some embodiments, the lobes 206 may be made from a material different from the upstanding wall 204.

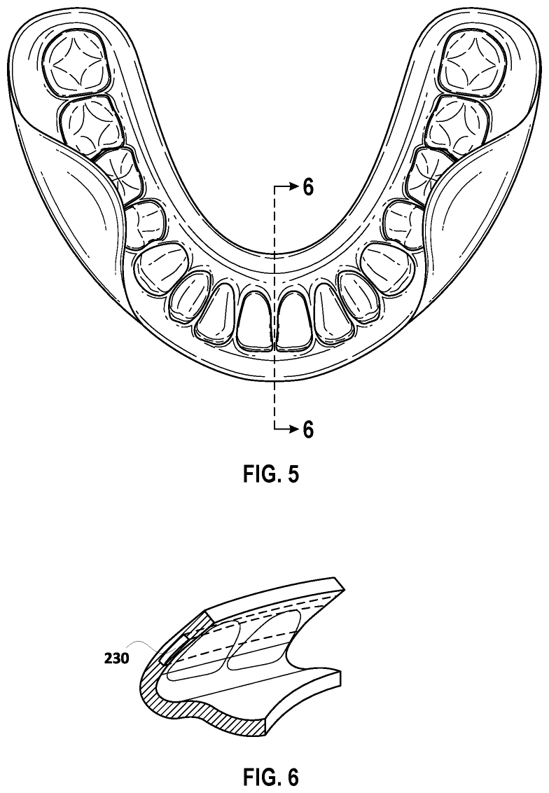

FIG. 5 shows a top view depicting the example intraoral apparatus 100, according to some embodiments. FIG. 6 shows a cross-sectional view along line 6-6 of the example intraoral apparatus 100, according to some embodiments. The intraoral apparatus 100 may have a wire 230 positioned within the upstanding wall 104. Although not shown in the figures, it is understood that the intraoral apparatus 200 may also have a wire positioned within the upstanding wall 204. The wire can be made of solid and bendable materials, such as metal, elastic materials, and fiberglass. The handle 220 may have a wire inserted inside as well.

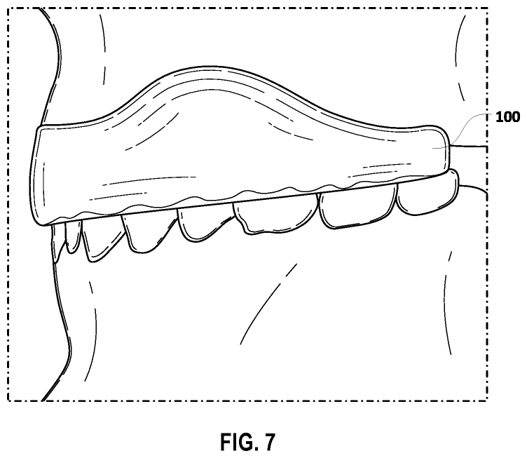

FIG. 7 shows a side view depicting the example intraoral apparatus 100 positioned within the user's mouth, according to some embodiments. The intraoral apparatus 100 can be placed in the user's upper jaw.

### Example Embodiments

In some embodiments, a method of reducing nasolabial folds can be used to prevent the nasolabial folds. The method starts by placing the intraoral apparatus inside the user's mouth. The intraoral apparatus is secured against the user's upper teeth. The method takes effect after regular usage of 6-8 hours daily for a certain period of time. For example, the user may start seeing improvements after three months for mild symptoms. For severe deep folds, it may take longer, up to a year, to see progress. The effect is significant, especially when the user is younger, e.g., before the age of 25. The user can obtain either the intraoral apparatus 100 under a lab setting or the intraoral apparatus 200 over the counter at a pharmacy.

In some embodiments, a method of reducing nasolabial folds can be used to maintain the visual appearance after an injectable filler treatment. The user can obtain either the intraoral apparatus 100 under a lab setting or the intraoral apparatus 200 over the counter at a pharmacy. The intraoral apparatus can be self-administered by the user overnight, which typically requires 6-8 hours on a daily basis.

The use of “including,” “comprising,” or “having” and variations thereof herein is meant to encompass the items listed thereafter and equivalents thereof as well as additional items. Unless specified or limited otherwise, the terms “affixed,” “associated,” “attached,” “connected,” “coupled” and “supported,” and variations thereof are used broadly and encompass both direct and indirect connections, supports, and couplings. It is to be understood that other embodiments may be utilized, and structural or logical changes may be made without departing from the scope of the present disclosure. Furthermore, terms such as “front,” “rear,” “top,” “bottom,” “left,” “right,” and the like are only used to describe elements as they relate to one another but are in no way meant to recite specific orientations of the apparatus, to indicate or imply necessary or required orientations of the apparatus, or to specify how the invention described herein will be used, mounted, displayed, or positioned in use.

What is claimed is:

1. An apparatus for reducing nasolabial folds, comprising:
an upstanding wall having a first surface facing a user's lip, the upstanding wall having two lobes positioned apart on either side of the upstanding wall, wherein the lobes extend upward from a top portion of the upstanding wall to an area where an upper lip and a gum join, wherein the lobes are of a size and thickness to lift a skin between each corner of a nose and each corner of the lip above from the gum, and wherein the first surface bulges outward at locations of the lobes; and
a tray having a second surface facing occlusal surfaces of lower teeth;
wherein the upstanding wall and the tray define a third surface having recesses for receiving upper teeth.

2. The apparatus of claim 1, further comprises a wire positioned within the upstanding wall, wherein the wire is of a substantially same length as the upstanding wall.

3. The apparatus of claim 1, wherein each of the lobes spans between first premolar and first molar of the upper teeth.

4. The apparatus of claim 1, wherein each of the lobes spans between canine and first molar of the upper teeth.

5. The apparatus of claim 1, wherein the lobes have a thickness between 0.1 cm to 3 cm.

6. The apparatus of claim 1, wherein the lobes have a thickness between 1 cm to 2 cm.

7. The apparatus of claim 1, wherein each of the lobes has a height between 1.5 cm to 3 cm from a top portion to a bottom portion of the upstanding wall.

8. An apparatus for reducing nasolabial folds, comprising:
an upstanding wall having a first surface facing a user's lip, the upstanding wall having two lobes positioned apart on either side of the upstanding wall, wherein the lobes extend upward from a top portion of the upstanding wall to an area where an upper lip and a gum join, wherein the lobes are of a size and thickness to lift a skin between each corner of a nose and each corner of the lip above from the gum, and wherein the first surface bulges outward at locations of the lobes; and
a handle coupled to a front portion of the upstanding wall.

9. The apparatus of claim 8, further comprises a wire positioned within the upstanding wall, wherein the wire is of a substantially same length as the upstanding wall.

10. The apparatus of claim 8, wherein each of the lobes spans between first premolar and first molar of the upper teeth.

11. The apparatus of claim 8, wherein each of the lobes spans between canine and first molar of the upper teeth.

12. The apparatus of claim 8, wherein the lobes have a thickness between 0.1 cm to 3 cm.

13. The apparatus of claim 8, wherein the lobes have a thickness between 1 cm to 2 cm.

14. The apparatus of claim 8, wherein each of the lobes has a height between 1.5 cm to 3 cm from the second surface to a top surface of the upstanding wall.

15. A method of reducing nasolabial folds, comprising:
administrating an intraoral apparatus configured to be worn daily, the intraoral apparatus comprising:
an upstanding wall having a first surface facing a user's lip and a second surface facing upper teeth, the upstanding wall having two lobes positioned apart on either side of the upstanding wall, wherein the lobes extend upward from a top portion of the upstanding wall to an area where an upper lip and a gum join, wherein the lobes are of a size and thickness to lift a skin between each corner of a nose and each corner of the lip above from the gum, and wherein the first surface bulges outward at locations of the lobes.

16. The method of claim 15, wherein the intraoral apparatus further comprises a handle coupled to a front portion of the upstanding wall.

17. The method of claim 15, wherein the intraoral apparatus further comprises a tray, wherein the upstanding wall and the tray define a third surface having recesses for receiving upper teeth.
